(19) United States
(12) Patent Application Publication
(10) Pub. No.: US 2026/0245202 A1
(43) Pub. Date: Aug. 20, 2026

(54) A METHOD FOR PROCESSING IMAGE, AN ELECTRONIC APPARATUS AND A COMPUTER READABLE STORAGE MEDIUM

(71) Applicant: MEDIT CORP., Seoul (KR)

(72) Inventor: Seon Yae PARK, Seoul (KR)

(21) Appl. No.: 18/994,010

(22) PCT Filed: Jul. 13, 2023
(86) PCT No.: PCT/KR2023/010061

§ 371 Date: Jan. 13, 2025

(30) Foreign Application Priority Data
Jul. 13, 2022 (KR) 10-2022-0086432
Jul. 12, 2023 (KR) 10-2023-0090639

(51) Int. Cl. G06T 7/00; A61C 5/00; G06T 7/11; G06T 7/70; G06T 17/20

(52) U.S. Cl. CPC G06T 7/0012; G06T 7/11; G06T 7/70; G06T 17/205; A61C 5/007; G06T 2207/20084; G06T 2207/30036; G06T 2215/16

(57) ABSTRACT

A method for processing an image, an electronic apparatus performing the same, and a computer readable storage medium storing the same may be provided. The method for processing an image includes receiving an intraoral image including maxillary scan data and mandibular scan data; calculating an occlusal distance to antagonist of the intraoral image based on an occlusal state of the intraoral image; and adjusting the occlusal distance to antagonist corresponding to the occlusal state of the intraoral image based on the calculating.

### TECHNICAL FIELD

The present disclosure relates to a method for processing an image, an electronic apparatus, and a computer readable storage medium.

### BACKGROUND ART

In cases where the temporomandibular joint is fatigued and temporomandibular joint disorder is caused due to improper occlusion, a splint, an orthodontic device, may be used to induce a stable central relation regardless of the malocclusion.

Since the splint should be inserted into an oral cavity, products should be manufactured in consideration of a distance and space in a patient's oral cavity to minimize additional processing and provide convenient products to the patient. An occlusal distance to antagonist between patient's arches should be confirmed according to each occlusal state, so an optimal thickness of a splint model may be determined and additional processing may be minimized.

However, there is a problem that a lot of data processing and time are required to calculate the occlusal distance to antagonist between the arches by reflecting the patient's oral characteristics with only a part of the occlusal state information.

### DISCLOSURE

### Technical Problem

The present disclosure attempts to provide a splint model for scan data while minimizing additional processing by displaying an occlusal state and an occlusal distance to antagonist together in response to the input occlusal state or occlusal distance to antagonist.

### Technical Solution

According to an exemplary embodiment, a method for processing an image includes: receiving an intraoral image including maxillary scan data and mandibular scan data; calculating an occlusal distance to antagonist (distance to antagonist) of the intraoral image based on an occlusal state of the intraoral image; and adjusting the occlusal distance to antagonist corresponding to the occlusal state of the intraoral image based on the calculating.

According to another exemplary embodiment, an electronic apparatus includes: a user interface device; a processor; and a memory storing instructions executable by the processor, in which the processor executes the instructions to: receive an intraoral image including maxillary scan data and mandibular scan data; calculate an occlusal distance to antagonist (distance to antagonist) of the intraoral image based on an occlusal state of the intraoral image; and adjust the occlusal distance to antagonist corresponding to the occlusal state of the intraoral image based on the calculation operation.

According to still another exemplary embodiment, there is provided a computer readable storage medium including computer readable instructions, in which the instructions cause the computer to: receive an intraoral image including maxillary scan data and mandibular scan data; calculate an occlusal distance to antagonist of the intraoral image based on an occlusal state of the intraoral image; and adjust the occlusal distance to antagonist corresponding to the occlusal state of the intraoral image based on the calculating.

### Advantageous Effect

According to the disclosed exemplary embodiments, it is possible to provide the splint model optimized for the patient by providing the occlusal distance to antagonist according to the occlusal state by reflecting the characteristics of the patient.

According to the disclosed exemplary embodiments, it is possible to reduce the time required to calculate the occlusal distance to antagonist according to the occlusal state and the data size by setting the effective range for the occlusal distance to antagonist.

### DESCRIPTION OF THE DRAWINGS

FIG. 1 is a diagram for describing an image processing system including an electronic apparatus according to an exemplary embodiment.

FIG. 2 is a block diagram illustrating a configuration of an electronic apparatus according to an exemplary embodiment.

FIG. 3 is a flowchart illustrating a method for processing an image of an electronic apparatus according to an exemplary embodiment.

FIGS. 4 and 5 are diagrams for describing scan data, maxillary scan data, and mandibular scan data according to an exemplary embodiment.

FIG. 6 is a diagram for describing an occlusal state of scan data according to an exemplary embodiment.

FIG. 7 is a flowchart illustrating a method for processing an image of an electronic apparatus according to an exemplary embodiment.

FIG. 8 is a diagram for describing a step of extracting a tooth region according to an exemplary embodiment.

FIG. 9 is a diagram for describing a step of adjusting an occlusal state investigation range and a step of calculating an intermediate occlusal distance to antagonist within the occlusal state investigation range according to an exemplary embodiment.

FIG. 10 is a diagram for describing a screen on which the occlusal state and the occlusal distance to antagonist are displayed according to an exemplary embodiment.

### MODE FOR INVENTION

Hereinafter, exemplary embodiments of the present invention will be described in detail with reference to the accompanying drawings so that those skilled in the art to which the present invention pertains may easily practice the present invention. The present invention may be implemented in various different forms and is not limited to exemplary embodiments provided herein.

Portions unrelated to the description will be omitted in order to obviously describe the present invention, and similar components will be denoted by the same reference numerals throughout the present specification.

In addition, the size and thickness of each component illustrated in the drawings are arbitrarily indicated for convenience of description, and the present invention is not necessarily limited to the illustrated those. In the drawings, the thickness of layers, films, panels, regions, etc., are exaggerated for clarity. In addition, in the accompanying drawings, thicknesses of some of layers and regions have been exaggerated for convenience of explanation.

In addition, it will be understood that when an element such as a layer, film, region, or substrate is referred to as being “on” another element, it can be directly on the other element or intervening elements may also be present. In contrast, when an element is referred to as being “directly on” another element, there are no intervening elements present.

In addition, when an element is referred to as being “on” a reference element, it can be positioned on or beneath the reference element, and is not necessarily positioned on the reference element in an opposite direction to gravity.

In addition, unless explicitly described to the contrary, the word “comprise”, and variations such as “comprises” or “comprising”, will be understood to imply the inclusion of stated elements but not the exclusion of any other elements.

Further, throughout the specification, the word “plane” refers to a view when a target is viewed from the top, and the word “cross section” refers to a view when a cross section of a target taken along a vertical direction is viewed from the side.

In addition, terms including an ordinal number such as first, second, or the like, used in the present disclosure may be used to describe various components. However, these components are not limited to these terms. The above terms are used solely for the purpose of distinguishing one component from another.

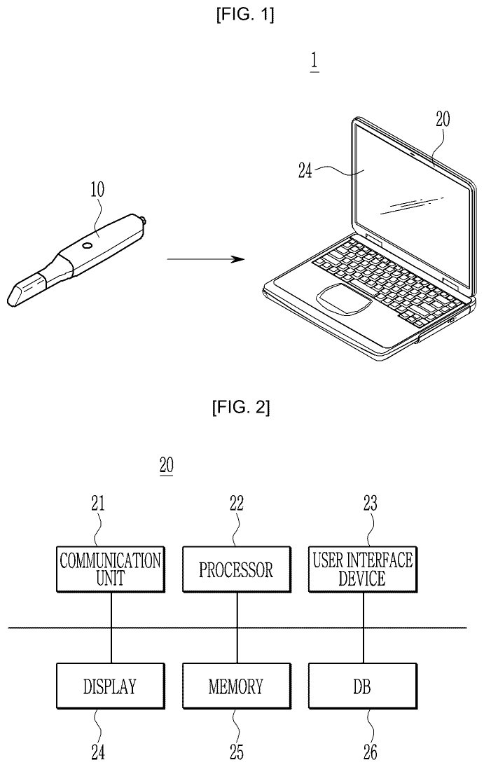

FIG. 1 is a diagram for describing an image processing system including an electronic apparatus according to an exemplary embodiment. FIG. 2 is a block diagram illustrating a configuration of an electronic apparatus according to an exemplary embodiment.

Referring to FIG. 1 and FIG. 2, a system 1 for processing an image may include a scanner 10 and an electronic apparatus 20.

In this specification, an ‘object’ is a capturing subject and may include a person, an animal, or a part thereof. For example, the object may include a part (an organ, an organ, etc.) of a body, a phantom, etc. In addition, for example, the object may include a plaster model modeling an oral cavity, a denture such as a denture or a prosthesis, a dentiform in a shape of teeth, etc. For example, the object may include a tooth, a gingiva, at least a portion of the oral cavity, and/or artificial structures (e.g., an orthodontic device including bracket and wire, dental restorations including implant, abutment, artificial teeth, inlay and onlay, and orthodontic auxiliary tools inserted into the oral cavity, etc.) that may be inserted into the oral cavity, the tooth or gingiva to which the artificial structures are attached, etc.

The scanner 10 may mean a device that acquires an image related to the object. The scanner 10 may mean a scanner 10 that acquires an intraoral image related to the oral cavity used for oral treatment. The scanner 10 may acquire at least one of a two-dimensional (2D) image and a three-dimensional (3D) image. In addition, the scanner 10 may acquire at least one 2D image of the oral cavity, and generate a 3D image (or a 3D model) of the oral cavity based on at least one acquired 2D image. In addition, the scanner may acquire at least one two-dimensional image of the oral cavity, and transmit the at least one two-dimensional image to the electronic apparatus 20.

The electronic apparatus 20 may also image a surface of at least one of the scanner 10 tooth model or tooth, a gingiva, and the artificial structures (e.g., the orthodontic device including the bracket and wire, the orthodontic auxiliary tools inserted into the oral cavity including the implant, the artificial teeth, and splint, etc.) insertable into the oral cavity, and for this purpose, may acquire surface information about the object as raw data.

The electronic apparatus 20 may generate the 3D image of the oral cavity based on at least one received 2D image. Here, the ‘3D image’ may be generated by three-dimensionally modeling the object based on the received raw data, and thus may be called a ‘3D model’. In addition, in the present disclosure, a model or image representing an object two-dimensionally or three-dimensionally may be collectively called an ‘image’.

For example, the scanner 10 may be an intraoral scanner having a form that may be inserted into the oral cavity, and according to the exemplary embodiment, the intraoral scanner may be a wired device or a wireless device, and the technical idea of the present disclosure is not limited to the form of the intraoral scanner.

According to an exemplary embodiment, the intraoral scanner may be a hand-held type scanner that can be held by hand and carried. The intraoral scanner may be inserted into the oral cavity, and scan teeth in a non-contact manner to obtain an image of the oral cavity including at least one tooth, and scan the inside of the patient's oral cavity using at least one image sensor (e.g., an optical camera, etc.).

According to an exemplary embodiment, the scanner 10 may be a table type scanner that may be used for dental treatment. The table type scanner may be a scanner that acquires the surface information on an object as the raw data by scanning the object using the rotation of the table. The table scanner may scan a surface of an object such as a plaster model or an impression model modeling the oral cavity.

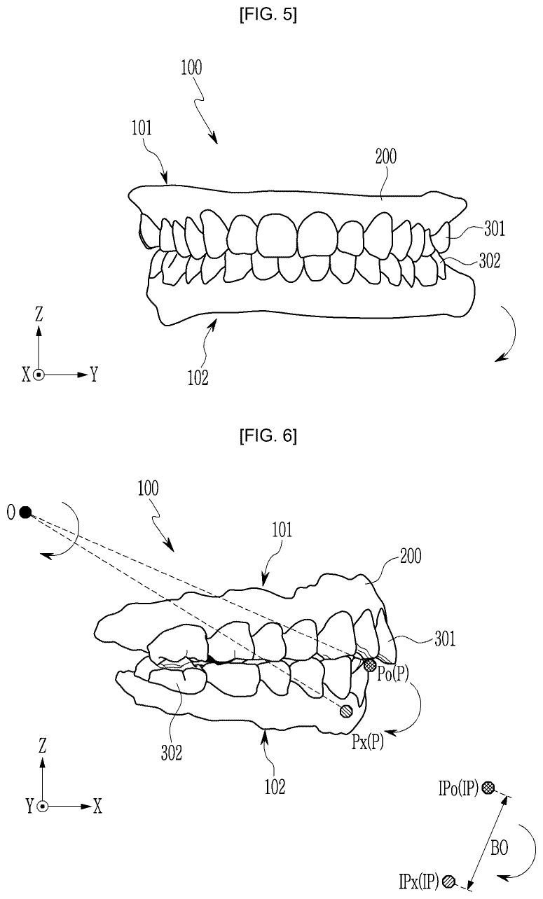

The electronic apparatus 20 may receive the raw data from the scanner 10 and process the received raw data to output a 3D image for the raw data. According to an exemplary embodiment, the output 3D image may be 3D image data for the splint for the received raw data. For ease of description, a specific description of the scan data is described later with reference to FIGS. 4 and 5.

The electronic apparatus 20 may be any electronic apparatus that is connected to the scanner via a wired or wireless communication network, and may receive a 2D image acquired by scanning the object from the scanner and generate, process, display, and/or transmit an image based on the received 2D image.

The electronic apparatus 20 may store and execute dedicated software to perform at least one operation of receiving, processing, storing, and/or transmitting the 3D image or the 2D image of the object. For example, the dedicated software may perform processing operations such as area extraction and area setting on the received scan data, and perform data selection, reference point adjustment, alignment, etc., based on the processing operations to perform at least one operation such as generation, storing, and transmitting the splint 3D image for the scan data such as the splint.

The electronic apparatus 20 may be a computing device such as a smart phone, a laptop computer, a desktop computer, a PDA, or a tablet PC, but is not limited thereto. In addition, the electronic apparatus 20 may exist in the form of a server (or server device) for processing intraoral images.

The electronic apparatus 20 may include a communication unit 21, a processor 22, a user interface device 23, a display 24, a memory 25, and a database 26. However, not all of the illustrated components are essential components. The electronic apparatus 20 may be implemented by more components than the illustrated components, or may be implemented by fewer components. The components will be described below.

The communication unit 21 may perform communication with an external device. Specifically, the communication unit 21 may be connected to a network by wire or wirelessly to perform communication with the external device. Here, the external device may be the scanner 10, a server, a smartphone, a tablet, a PC, etc.

The communication unit 21 may include a communication module that supports one of various wired and wireless communication methods. For example, the communication module may be in the form of a chipset, or may be a sticker/barcode (e.g., a sticker including an NFC tag), etc., including information necessary for communication. In addition, the communication module may be a short-range communication module or a wired communication module.

For example, the communication unit 21 may support at least one of wireless LAN, wireless fidelity, Wi-Fi direct, Bluetooth, Bluetooth low energy, wired LAN, near field communication, Zigbee, Infrared data association (IrDA), 3G, 4G, and 5G. In an exemplary embodiment, the scanner 10 may transmit the acquired raw data to the electronic apparatus 20 through the communication module. The image data acquired by the scanner may be transmitted to the electronic apparatus 20 connected through the wired or wireless communication network.

The processor 22 controls the overall operation of the electronic apparatus 20 and may include at least one processor, such as a CPU. The processor 22 may include at least one specialized processor corresponding to each function, or may be a processor integrated into one.

The processor 22 may receive the raw data through the communication unit 21. For example, the processor 22 may receive the raw data from the scanner 10 through the communication unit 21. In this case, the processor 22 may generate the 3D image data (e.g., surface data, mesh data, etc.) that represents the shape of the surface of the object three-dimensionally based on the received raw data. Hereinafter, the scan data that becomes the calculation target of the electronic apparatus 20 may include the 3D image data.

The processor 22 may receive library data from the external device through the communication unit 21. The library data may be data pre-stored in the electronic apparatus 20 or the raw data or the 3D image data acquired through the external device, but is not limited thereto. Here, the external device may be a camera capable of capturing pictures or videos, or an electronic apparatus having a camera function. In addition, the external device may be an intraoral scanner capable of scanning the inside of a patient's mouth.

The processor 22 may control the user interface device 23 or the display 24 to receive a predetermined command or data from a user. The processor 22 may execute a program stored in the memory 25, read an image, data, or file stored in the memory 25, or store a new file in the memory 25. The processor 22 may execute instructions stored in the memory 25. The stored program may include, but is not limited to, dedicated software.

The processor 22 may perform a calculation operation on mesh data, data, etc., included in the scan data. For example, the processor 22 may perform operations such as calculating an average of normal vectors of the mesh data constituting the scan data, generating image intersections, calculating multiple distance values between data, or selecting the minimum of a plurality of generated distance values, but the operations of the processor 22 are not limited to the above examples.

According to an exemplary embodiment, the processor 22 may select a portion of the mesh data to be the target of the operation in the mesh data. The processor 22 may extract a normal vector for a selected portion of the mesh data, generate a straight line that proceeds in the direction of the normal vector with the selected mesh as a starting point, and generate the image intersections for the generated straight line.

The image intersection may be generated by utilizing a ray tracing algorithm. For example, the image intersection may be generated by finding intersections by passing a straight line such as a light ray through the scan data, but is not limited thereto.

The processor 22 may recognize an object in the scan data, extract a portion of an area, or calculate the area or volume of the recognized object or the extracted area. For example, the processor 22 may recognize the type of teeth by using a curvature information, cusp information, etc., of the scan data, or distinguish a space between teeth. According to an exemplary embodiment, the recognition operations of the processor 22 are not limited to the examples of utilizing the information, and the recognition operations may be performed through the inference of the object recognition artificial intelligence algorithm. The cusp information may include the number and arrangement of cusp points where opposing teeth in the scan data come into contact.

The processor 22 may calculate an occlusal distance to antagonist according to the occlusal state of the scan data. The scan data may include maxillary scan data and mandibular scan data, and the processor 22 may calculate the occlusal distance to antagonist of the maxillary scan data and the mandibular scan data corresponding to the occlusal state of the maxillary scan data and the mandibular scan data.

The occlusal distance to antagonist indicates the distance to the opposing teeth according to a specific occlusal state between the maxilla and the mandible. According to an exemplary embodiment, the occlusal distance to antagonist may mean the distance to antagonist among the distances between the maxillary structures and the mandibular structures in a specific occlusal state.

The processor 22 may adjust the occlusal state investigation range. For example, the processor 22 may adjust the occlusal state investigation range based on the calculated occlusal distance to antagonist and the preset distance to antagonist effective range. The processor 22 may adjust the occlusal state investigation range to be narrower than the occlusal state corresponding to the calculated occlusal distance to antagonist when the calculated occlusal distance to antagonist is not included in the distance to antagonist effective range, but the adjustment operation is not limited to the above example.

The processor 22 may adjust the distance to antagonist effective range. The processor 22 may adjust the occlusal state investigation range and calculate the occlusal distance to antagonist for the intermediate occlusal state repeatedly to perform calculation for the occlusal state not investigated within the occlusal state investigation range, while adjusting the distance to antagonist effective range.

According to an exemplary embodiment, the occlusal state may be expressed in the form of bite opening, and a specific description will be described later in the description of FIG. 6.

The processor 22 may search for data that requires calculation within the occlusal state investigation range. For example, the processor 22 may search for the intermediate occlusal state that requires calculation of the occlusal distance to antagonist in significant digit units within the adjusted occlusal state investigation range, but the search operation is not limited to the above example.

The user interface device 23 may mean a device that receives data from a user to control the electronic apparatus 20. The display 24 may include an output device for displaying a result image according to the operation of the electronic apparatus 20 or the 3D image output from the electronic apparatus 20.

The user interface device 23 may include, for example, an input device such as a mouse, a joystick, an operation panel, a touch sensitive panel that receives user input, and the display 24 may include a display panel that displays a screen, etc.

The memory 25 may store software or a program, and the stored software or program may be dedicated software, but is not limited thereto. The memory 25 may store at least one command for executing an operation method of the electronic apparatus 20 that calls scan data, adjusts the occlusion of the scan data, calculates and displays the bite opening and the occlusal distance to antagonist according to the occlusal state of the scan data, and the calculated bite opening and the occlusal distance to antagonist together.

The database 26 may store data and a dataset for training an artificial intelligence algorithm of dedicated software, and may provide data for training according to a request of the dedicated software. The artificial intelligence algorithm may train the training data of teeth stored in the database 26 using a deep learning method and distinguish the characteristics of data representing teeth. Meanwhile, the dedicated software may use the extracted or recognized tooth region data when performing an occlusal plane alignment step, an inner setting step, an outline designation step, etc., which will be described later, by extracting maxillary tooth region data and mandibular tooth region data from scan data or recognizing objects according to tooth characteristics.

In the present disclosure, the artificial intelligence (AI) means a technology that imitates human learning ability, reasoning ability, and perception ability and implements them with a computer, and may include the concepts of machine learning and symbolic logic. The machine learning (ML) may be an algorithm technology that classifies or trains the characteristics of input data on its own. The technology of the artificial intelligence may analyze input data as the machine learning algorithm, train the results of the analysis, and make the judgment or prediction based on the results of the training. In addition, technologies that imitate the cognitive and judgment functions of the human brain by utilizing the machine learning algorithm may also be understood as part of the category of the artificial intelligence. For example, the fields of technology of linguistic understanding, visual understanding, inference/prediction, knowledge expression, and motion control may be included.

In this disclosure, the machine learning may mean a process of training a neural network model using experience in processing data. Through the machine learning, the computer software may mean improving its own data processing ability. A neural network model is constructed by modeling correlations between data, and the correlations may be expressed by multiple parameters. The neural network model extracts and analyzes features from given data to derive correlations between data, and repeats the process to optimize the parameters of the neural network model, which may be called the machine learning.

For example, the neural network model may train a mapping (correlation) between inputs and outputs for data given as input-output pairs. Alternatively, even when only the input data is given, the neural network model may derive regularities between the given data and train the relationship.

In the present disclosure, the artificial intelligence training model, the machine learning model, or the neural network model may be designed to implement a human brain structure on a computer, and may include a plurality of network nodes that simulate neurons of a human neural network and have weights. The plurality of network nodes may simulate synaptic activity of neurons that exchange signals through synapses, and thus may have a connection relationship between each other. In the artificial intelligence learning model, the plurality of network nodes may be located in layers of different depths and may exchange data according to the convolution connection relationship.

Although the database 26 is illustrated as being included in the electronic apparatus 20 in the drawing, it is not limited thereto and may be arranged in the form of a server (or server device) or the like outside the electronic apparatus 20 to provide data for training and store training results.

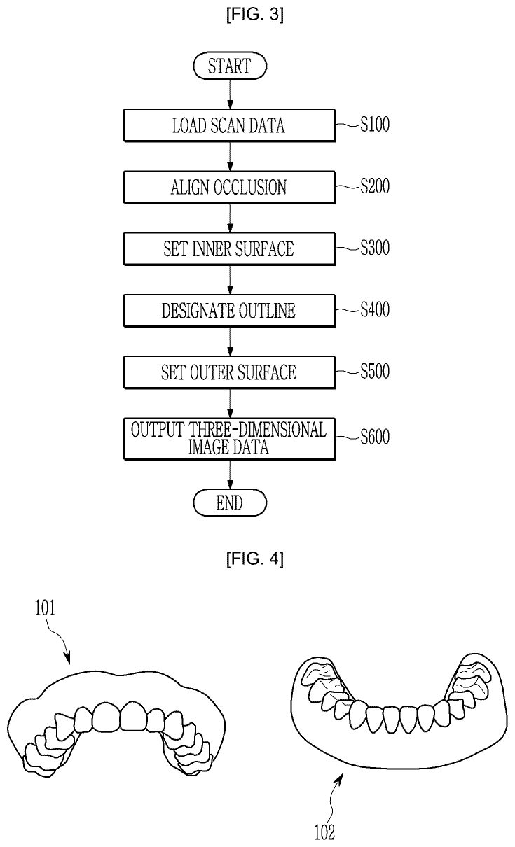

FIG. 3 is a flowchart illustrating a method for processing an image of an electronic apparatus according to an exemplary embodiment. FIGS. 4 and 5 are diagrams for describing scan data, maxillary scan data, and mandibular scan data according to an exemplary embodiment.

Referring to FIGS. 1 to 5, the electronic apparatus 20 loads scan data 100 (S100). The electronic apparatus 20 loads the scan data 100 generated based on the image received from the external device including the scanner 10, etc., through the communication unit 21.

The electronic apparatus 20 loads scan data 100 processed based on the received image or pre-stored in the processor 22 or the user interface device 23, and may display the loaded scan data 101 through the display 24.

The scan data 100 may be the 2D image of the object, the 3D model representing the object in three dimensions, or the 3D image data, and specifically, may be a 3D intraoral model. According to an exemplary embodiment, the intraoral images in FIGS. 4 and 5 correspond to the scan data 100 and are 2D or 3D expressions of the objects of the scan data 100, and may include a maxillary pre-preparation (prep) image, a maxillary prep image, a mandibular pre-prep image, a mandibular prep image, an occlusal image including a maxillary-related image and a mandibular-related image, as in the scan data 100 described below.

In the present disclosure, the prep may mean a series of preparatory processes for removing a portion of the enamel and dentin of the teeth so as to prevent interference between natural teeth and splints when performing prosthetics such as crowns and prostheses.

A “3D intraoral model” may mean a model that three-dimensionally models the oral cavity based on the raw data acquired by the scanning operation of the scanner. In addition, the “3D intraoral model” may mean a structure that is three-dimensionally modeled based on the data acquired by scanning an object such as a tooth, an impression, and an artifact. The 3D intraoral model is generated by modeling the internal structure of the oral cavity in three dimensions, and may be called a 3D scan model, a 3D model, or a tooth model. For example, a format of the 3D intraoral model may be one of standard triangle language (STL), OBJ, and polygon file formats, and is not limited to the above examples. In addition, the 3D intraoral model may include information such as geometric information, color, texture, and a material for a 3D shape.

In addition, the “polygon” may mean a polygon which is the smallest unit used when expressing the 3D shape of the 3D intraoral model. For example, the surface of the 3D intraoral model may be expressed as triangular polygons. For example, a polygon may be composed of at least three vertices and one face. A vertex may include information such as location, color, and normal. A mesh may be an object in a 3D space created by gathering multiple polygons. As the number of polygons representing the 3D intraoral model increases, the object may be expressed in detail.

The scan data 100 may include at least one of the maxillary scan data 101 and the mandibular scan data 102. Specifically, the scan data 100 may load any one of the maxillary pre-prep data, the maxillary prep data, the mandibular pre-prep data, the mandibular prep data, and the occlusal data including the maxillary-related data and the mandibular-related data. In the present disclosure, the prep means a series of preparatory processes for removing a portion of enamel and dentin of the tooth so as to prevent interference between the natural tooth and the prosthesis when performing prosthetics such as crowns and bridges. The prep data may be data in which the enamel and dentin of the tooth are removed through the preparatory process, and the pre-prep data may be data before a portion of the enamel and dentin of the tooth are removed through the preparatory process.

The scan data 100 may include gingival area 200, maxillary tooth region data 301, and mandibular tooth region data 302, which are arranged in the maxillary scan data 101 and the mandibular scan data 102.

The electronic apparatus 20 may load at least one of the maxillary scan data 101, the mandibular scan data 102, and the occlusal data including the maxillary scan data 101 and the mandibular scan data 102.

The electronic apparatus 20 analyzes and aligns the shape of the received scan data 100 (S200). In the corresponding step, an occlusal plane and a midline for the scan data 100 are set, and the electronic apparatus 20 may automatically align the scan data 100, the maxillary scan data 101, or the mandibular scan data 102 according to the occlusal plane, and may display the left and right alignment by a midline through the display 24.

In addition, at the corresponding stage, the user may manually designate a reference point on the scan data 100 to set the front direction and the occlusal plane of the scan data 100, and align the scan data 100 along the set occlusal plane. For example, the user may select some data of the scan data 100 through the user interface device 23 at the corresponding step, and align the scan data 100 with the selected data as a reference point.

The electronic apparatus 20 sets the inner surface of the splint for the aligned scan data 100 (S300).

At the corresponding step, the electronic apparatus 20 may designate the direction in which the splint is to be inserted by considering the undercut of the aligned scan data 100. For example, when manufacturing the splint, the electronic apparatus 20 may calculate the area of the tooth region in the scan data 100, and designate the direction in which the splint is to be inserted by considering the undercut and block out according to the direction in which the splint is to be inserted. The insertion efficiency and retention force of the splint may be improved by designating the insertion direction of the splint as described above.

Based on the inner surface offset distance, the surface smoothness, etc., input from the user interface device 23, the electronic apparatus 20 may set the inner surface of the splint to be output. The inner surface offset distance may mean a separation distance in the normal direction between the scan data 100 and the inner surface of the splint. The surface smoothness may mean the roughness of the inner surface of the splint.

The electronic apparatus 20 designates the outline of the splint for the automatically aligned scan data 100 (S400).

Based on the buccal height, the lingual height, etc., input from the user interface device 23, the electronic apparatus 20 may designate the outline of the splint to be output. For example, when manufacturing the splint, the buccal height is a height of the outer wall of the tooth facing a cheek based on a lower surface of the tooth region, and for example, the buccal height may mean a height formed along the outer wall of the tooth based on the bottom surface of the tooth region of the maxillary scan data 101. The higher the buccal height, the closer the buccal outline formed is to the gingiva. The lingual height is a height of the inner wall of the tooth facing a tongue based on a bottom surface of the tooth region, and for example, the lingual height may mean a height formed along the inner wall of the tooth based on the bottom surface of the tooth region of the maxillary scan data 101. The higher the lingual height, the closer the lingual outline formed is to the gingiva.

The electronic apparatus 20 sets the outer surface of the splint for the aligned scan data 100 (S500).

Based on the thickness, the surface smoothness, etc., input from the user interface device 23, the electronic apparatus 20 may designate the outer surface of the splint to be output. The electronic apparatus 20 may form the 3D image for the splint by setting the thickness of the splint in the occlusal direction based on the predetermined occlusal thickness. For example, when manufacturing the splint, the thickness may mean the thickness from the inner surface of the splint in the buccal/lingual direction. The surface smoothness may mean the roughness of the outer surface of the splint. The predetermined occlusal thickness may mean the maximum thickness value that the splint extends in the occlusal direction.

The electronic apparatus 20 generates the 3D image data including the splint through the information set and designated in steps S300 to S500 (S600). The 3D image of the generated splint may be transmitted to the external device through the communication unit 21 and output to the splint. The external device may be a 3D printer, but is not limited to the above example according to an exemplary embodiment.

According to an exemplary embodiment, the electronic apparatus 20 may perform steps S200 to S500 at once without an intermediate input from the user. The electronic apparatus 20 loads the scan data 100 (S100), receives inputs, such as the inner surface offset distance, the surface smoothness, the buccal height, the lingual height, and the thickness, from the user, and automatically performs steps S200 to S500 without intermediate intervention from the user to generate the 3D image data for the splint (S600).

According to an exemplary embodiment, the electronic apparatus 20 may automatically perform steps S200 to S500 without user intervention by utilizing the inner surface offset distance, the surface smoothness, the buccal height, the lingual height, the thickness, etc., stored in the memory 25.

The electronic apparatus 20 may automatically perform steps S200 to S500 without the user intervention, thereby reducing the time required for the splint manufacturing.

According to an exemplary embodiment, the electronic apparatus 20 may generate the 3D image data for the splint through an inference operation of the artificial intelligence algorithm without a separate input other than the loaded scan data 100 (S600). The artificial intelligence algorithm may perform training on the splint corresponding to the plurality of scan data before the inference operation, and perform the inference operation related to the 3D image data of the splint suitable for the loaded scan data 100.

According to an exemplary embodiment, the electronic apparatus 20 may adjust the occlusal state or the occlusal distance to antagonist between arches (distance to antagonist) in each step of steps S300 to S500 through the user input regarding the occlusal state or the occlusal distance to antagonist between the arches between the maxillary scan data 101 and the mandibular scan data 102 in the scan data 100. During the adjustment operation, the electronic apparatus 20 may perform the calculation operation on the scan data 100 to display occlusal state or the occlusal distance to antagonist between the arches together.

Through the adjustment and calculation operations, the user may manufacture the splint by considering the distance and space in the patient's oral cavity, and through this, the electronic apparatus 20 may minimize additional processing after manufacturing and provide the splint optimized for the patient. The adjustment and calculation display operations are described later in the description of FIGS. 6 to 10.

FIG. 6 is a diagram for describing the occlusal state of the scan data according to an exemplary embodiment.

Referring to FIG. 6, the electronic apparatus 20 may adjust the occlusal state between the maxillary scan data 101 and the mandibular scan data 102 centered on a virtual hinge O by considering the actual movement of the maxilla and the mandible. For example, the occlusal state of the scan data 100 may be adjusted while the maxillary scan data 101 is fixed and the mandibular scan data 102 is rotated around an axis extending from the hinge O in the second direction Y.

According to an exemplary embodiment, the electronic apparatus 20 may adjust the occlusal state of the maxilla and the mandible by rotating a midpoint P between the central incisors in the mandibular scan data 102 in the second direction Y axis according to the user input, and the position of the midpoint P between the central incisors may represent the occlusal state between the maxillary scan data 101 and the mandibular scan data 102.

According to an exemplary embodiment, the electronic apparatus 20 may calculate the occlusal state of the scan data 100 in the form of the bite opening BO. The bite opening BO is an index for explaining the vertical relationship of the occlusal state between the maxilla and the mandible, and may be calculated as a straight-line distance by calculating the change in the position of the incisal guide pin IP corresponding to the occlusal state assuming that the oral cavity is mounted on an articulator. According to an exemplary embodiment, since the bite opening corresponds to the position of the midpoint P, the occlusal state may be expressed as the bite opening.

For example, when the maxillary scan data 101 and the mandibular scan data 102 are in the occlusal contact relationship, the reference position of the midpoint P is Po, and the reference position of the corresponding incisor guide pin IP is IPo, and when the position of the midpoint P changes from the reference position Po to Px and the position of the corresponding incisor guide pin IP changes from the reference position IPo to IPx, the bite opening according to the changed occlusal state of the scan data 100 is BO, which is a straight line distance between IPo and IPx.

According to an exemplary embodiment, the electronic apparatus 20 may adjust the occlusal state of the maxilla and the mandible by rotating the midpoint between the central incisors in the maxillary scan data 101 along the second direction Y axis according to the user input, and the position of the midpoint between the central incisors may represent the occlusal state between the maxillary scan data 101 and the mandibular scan data 102.

According to an exemplary embodiment, when the electronic apparatus 20 receives the bite opening through the user interface device 23, the electronic apparatus 20 displays the occlusal state of the maxilla and the mandible corresponding to the bite opening and the occlusal distance to antagonist between the arches corresponding to the bite opening according to the operation of the electronic apparatus 20 described below through the display 24. In addition, when the electronic apparatus 20 receives the occlusal distance to antagonist between the arches through the user interface device 23, the electronic apparatus 20 displays the bite opening corresponding to the occlusal distance to antagonist between the arches and the occlusal state of the maxilla and the mandible corresponding to the bite opening according to the operation of the electronic apparatus 20 described below through the display 24.

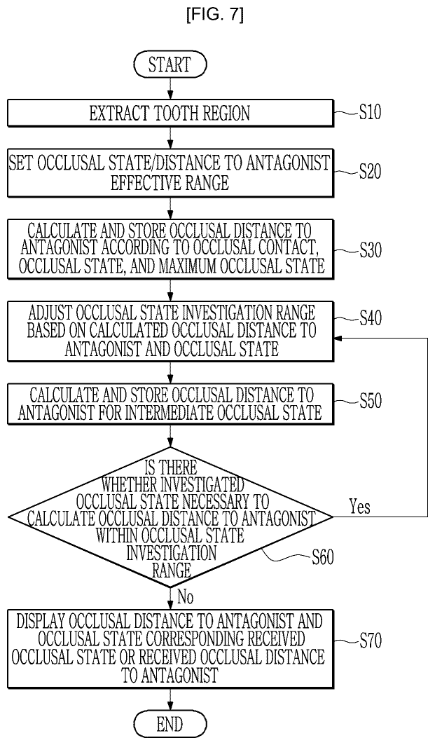

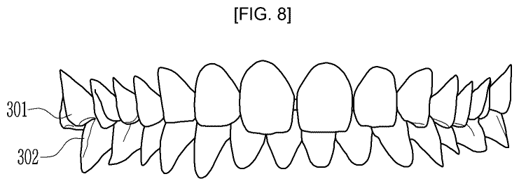

FIG. 7 is a flowchart illustrating a method for processing an image of an electronic apparatus according to an exemplary embodiment.

Referring to FIGS. 1 to 7, the electronic apparatus 20 may display the occlusal state and the occlusal distance to antagonist of the calculated scan data 100 while adjusting the occlusion between the maxillary scan data 101 and the mandibular scan data 102 according to the user input between steps S100 and S600.

The electronic apparatus 20 may extract the tooth region from the scan data 100 (S10).

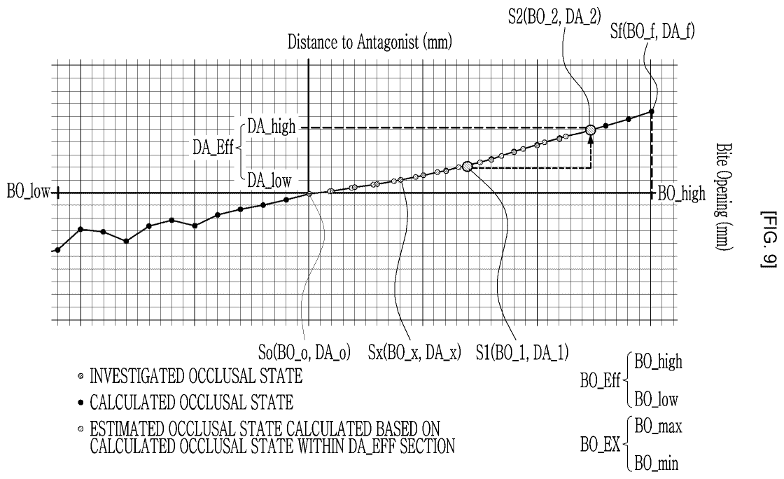

Additionally referring to FIG. 8, the electronic apparatus 20 may recognize and extract the tooth regions 301 and 302 from the scan data 100. The electronic apparatus 20 may recognize the tooth regions 301 and 302 by using curvature information, cusp information, etc., or may recognize and extract the tooth regions 301 and 302 through an object recognition artificial intelligence algorithm, and may identify and recognize the tooth number for each tooth in the tooth regions 301 and 302 by performing an inference operation on the tooth regions 301 and 302. The electronic apparatus 20 may reduce the time required for calculating the occlusal distance to antagonist and the data size by removing unnecessary structures such as gingiva by extracting the tooth region.

The electronic apparatus 20 sets the occlusal state effective range and the distance to antagonist effective range for the scan data 100 (S20).

The electronic apparatus 20 may set the occlusal state effective range and the distance to antagonist effective range that are easy to manufacture the splint, under the premise that the splint is inserted into the oral cavity. The occlusal state effective range may be −30.00 mm to 30.00 mm. The distance to antagonist effective range may be −30.00 mm to 30.00 mm, preferably 00.00 to 15.00 mm, and more preferably 0.00 mm to 5.00 mm, but the technical idea of the present disclosure is not limited to the above numerical values and significant digit units.

The electronic apparatus 20 calculates and stores the occlusal distance to antagonist according to the occlusal contact occlusal state, and the maximum occlusal state (S30).

The electronic apparatus 20 may select the occlusal contact occlusal state and the maximum occlusal state to effectively and efficiently adjust the occlusal state investigation range for splint manufacturing. According to an exemplary embodiment, the occlusal contact occlusal state is a reference state in which the maxillary scan data 101 and the mandibular scan data 102 are in the occlusal contact relationship, and the maximum occlusal state may be the state corresponding to the maximum value within the occlusal state effective range, but is not limited thereto.

According to one exemplary embodiment, the electronic apparatus 20 may calculate the occlusal distance to antagonist based on the extracted tooth regions 301 and 302. Hereinafter, steps S30 to S70 are described based on the operation for the mandibular tooth region 302, including generating a normal vector for the mandibular tooth region 302, but the technical idea of the present disclosure is not limited thereto, and the description of the operations of the electronic apparatus 20 in steps S30 to S70, which are described later, including generating the normal vector for the maxillary tooth region 301, may be applied.

According to one exemplary embodiment, the electronic apparatus 20 may extract the normal vector corresponding to each of the M meshes for the mandibular tooth region 302 including the M meshes.

The electronic apparatus 20 may generate a straight line that proceeds in the direction of the normal vector using the M meshes as a starting point, and may obtain the intersection of the generated straight line and the maxillary tooth region 301. According to an exemplary embodiment, the generated straight line may proceed using the vertex, the midpoint, etc., of the mesh as a starting point, but is not limited thereto. The electronic apparatus 20 calculates the distance values between the plurality of intersections corresponding to the M meshes, may set the minimum of the calculated plurality of distance values as the occlusal distance to antagonist according to the occlusal state, and may be determined as in the following Equation 1.

$$da = {min}_{1 \leq = m = < M} dm \qquad ( Equation 1 )$$

In the Equation 1 above, da is the occlusal distance to antagonist according to the occlusal state, and dm is the distance between an mth mesh and the corresponding mth intersection.

For example, the electronic apparatus 20 may calculate an xth distance between an xth intersection corresponding to an arbitrary xth mesh among the M meshes, and when the xth distance is the minimum of the plurality of distance values corresponding to the M meshes, the electronic apparatus 20 may select the xth distance as the occlusal distance to antagonist da according to the occlusal state.

According to one exemplary embodiment, the electronic apparatus 20 may select some of the M meshes included in the mandibular tooth region 302, extract the normal vector of the selected mesh, generate the straight line that proceeds in the direction of the normal vector with the selected mesh as the starting point, and obtain the intersection of the generated straight line and the maxillary tooth region 301. In an exemplary embodiment, some of the selected M meshes may be the tooth region, but are not limited thereto.

The electronic apparatus 20 may calculate the occlusal distance to antagonist according to the occlusal state through the selected mesh and the intersection. For example, the electronic apparatus 20 may select the mesh in a manner that excludes a waste area (a small area that is not continuous, an area scanned in the opposite direction to the occlusal direction, a gingival area, etc.) unnecessarily acquired during the scanning process for generating the scan data 100. Through the selection process, the electronic apparatus 20 may perform the calculation on the effective area in the scan data 100 to increase the time and efficiency required for calculating the occlusal distance to antagonist.

The electronic apparatus 20 may store the bite opening corresponding to the occlusal distance to antagonist da and the occlusal distance to antagonist da calculated according to an exemplary embodiment.

The electronic apparatus 20 may store the bite opening of the occlusal contact occlusal state and the occlusal distance to antagonist corresponding thereto together, and the bite opening of the maximum occlusal state corresponding to the maximum value within the occlusal state effective range and the occlusal distance to antagonist corresponding thereto together.

The electronic apparatus 20 adjusts the occlusal state investigation range based on the calculated occlusal distance to antagonist and the occlusal state (S40).

Referring additionally to FIG. 9, the electronic apparatus 20 may adjust an occlusal state investigation range BO_EX based on the calculated occlusal contact occlusal state So and maximum occlusal state Sf. According to an exemplary embodiment, the occlusal contact occlusal state So may correspond to a reference state in which the maxilla and the mandible are in occlusal contact, and the maximum occlusal state Sf may correspond to BO_high, which is the maximum value within the occlusal state effective range BO_Eff.

According to an exemplary embodiment, when the occlusal contact occlusal state So is a bite opening of BO_o and the occlusal distance to antagonist is calculated as DA_o, and the DA_o is smaller than a minimum value DA_low of the minimum distance effective range DA_Eff, the electronic apparatus 20 sets a minimum value BO_min of the occlusal state investigation range BO_EX to BO_o and does not perform the occlusal distance to antagonist calculation operation for the occlusal state less than the BO_o.

In addition, when the occlusal distance to antagonist of the maximum occlusal state Sf corresponding to BO_high, which is the maximum value within the occlusal state effective range BO_Eff, is calculated as DA_f and the DA_f is greater than the maximum value DA_high of the minimum distance effective range DA_Eff, the electronic apparatus 20 sets the maximum value BO_max of the occlusal state investigation range BO_EX to BO_high and does not perform the occlusal distance to antagonist calculation operation for the occlusal state exceeding BO_high.

The electronic apparatus 20 calculates and stores the intermediate occlusal distance to antagonist for the intermediate occlusal state within the occlusal state investigation range BO_EX (S50).

The electronic apparatus 20 may calculate the first intermediate occlusal distance to antagonist corresponding to the first intermediate occlusal state S1 between the maximum value BO_max and the minimum value BO_min of the occlusal state investigation range BO_EX. The bite opening of the first intermediate occlusal state S1 is BO_1, and the first intermediate occlusal distance to antagonist of the first intermediate occlusal state S1 may be calculated as DA_1. According to an exemplary embodiment, the electronic apparatus 20 may store BO_1 and DA_1 together.

The calculation process of the first intermediate occlusal distance to antagonist may be replaced with the description of the calculation of the occlusal distance to antagonist in step S30, and may also be applied to the calculation process of other intermediate occlusal distances to antagonists within the occlusal state investigation range BO_EX in this specification.

The electronic apparatus 20 confirms whether there is the investigation occlusal state that requires the calculation of the occlusal distance to antagonist within the occlusal state investigation range BO_EX (S60).

The electronic apparatus 20 may search for the investigation occlusal state that requires the calculation of the occlusal distance to antagonist within the occlusal state investigation range BO_EX. According to an exemplary embodiment, the electronic apparatus 20 may search for the bite opening in significant digit units within the occlusal state investigation range BO_EX and confirm the investigation occlusal state for which the occlusal distance to antagonist is not calculated within the occlusal state investigation range BO_EX. According to an exemplary embodiment, the electronic apparatus 20 may investigate the occlusal state within the occlusal state investigation range BO_EX in the significant digit unit of 0.01 mm, but the technical idea of the present disclosure is not limited to the examples of the significant digit unit.

The electronic apparatus 20 performs an operation of repeating steps S40 to S60 when it is determined that there is the investigation occlusal state. According to an exemplary embodiment, the electronic apparatus 20 may adjust the minimum distance effective range DA_Eff during the operation of repeating steps S40 to S60. According to an exemplary embodiment, the electronic apparatus 20 may adjust the minimum distance effective range DA_Eff from −30.00 mm to 30.00 mm to 00.00 to 15.00 mm, and may adjust the minimum distance effective range DA_Eff from 00.00 to 15.00 mm to 0.00 mm to 5.00 mm. The numerical range for the minimum distance effective range DA_Eff that changes according to the adjustment operation may change according to the user input, and the technical idea of the present disclosure is not limited to the examples of the above numerical ranges.

The electronic apparatus 20 may adjust, through the repeated operations, the bite opening value BO_2 of the second intermediate occlusal state S2 corresponding to the maximum value DA_high within the minimum distance effective range DA_Eff to the maximum value BO_max of the occlusal state investigation range BO_EX, and adjust the bite opening value BO_o of the occlusal contact occlusal state So corresponding to the minimum value DA_low within the minimum distance effective range DA_Eff to the minimum value BO_min of the occlusal state investigation range BO_EX.

For example, the electronic apparatus 20 may search for, within the occlusal state investigation range BO_EX in significant digit units, the xth intermediate occlusal state Sx with an arbitrary bite opening value BO_x for which the occlusal distance to antagonist is not calculated as the investigated occlusal state, calculate DA_x, which is the xth intermediate occlusal distance to antagonist value corresponding to the xth intermediate occlusal state Sx, and store the BO_x and DA_x together.

The electronic apparatus 20 receives the occlusal state or the occlusal distance to antagonist, and displays the occlusal distance to antagonist corresponding to the received occlusal state or the occlusal state corresponding to the received occlusal distance to antagonist (S70).

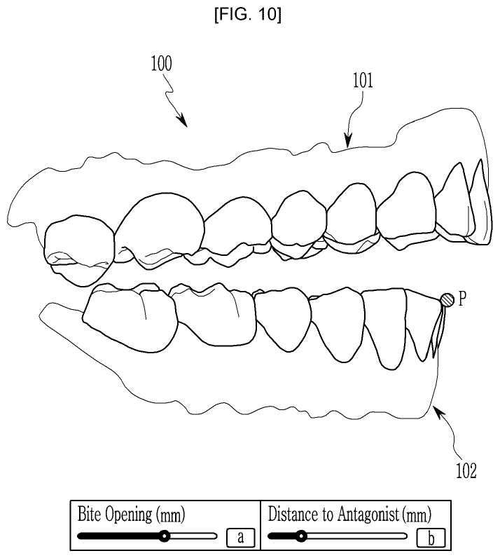

Referring additionally to FIG. 10, according to an exemplary embodiment, when the electronic apparatus 20 determines that there is no the investigated occlusal state, it may receive and load the bite opening value or the occlusal distance to antagonist value pre-stored in the electronic apparatus 20 without input from a separate user interface device 23.

According to an exemplary embodiment, when the electronic apparatus 20 determines that there is no investigated occlusal state, it may receive the bite opening value a or the occlusal distance to antagonist value b through the user interface device 23.

When the arbitrary bite opening value a is received, the electronic apparatus 20 searches for the corresponding stored bite opening within the occlusal state investigation range BO_EX, adjusts the occlusal distance to antagonist of the scan data 100 corresponding to the stored bite opening value a to b to be displayed together.

When the arbitrary occlusal distance to antagonist value b is received, the electronic apparatus 20 searches for the corresponding stored occlusal distance to antagonist value b within the minimum distance effective range DA_Eff, and adjusts the bite opening of the scan data 100 corresponding to the stored occlusal distance to antagonist value b to a to be displayed together.

The electronic apparatus 20 may display the scan data 100 in which the position of the midpoint P between the central incisors is adjusted according to the input bite opening value a or the occlusal distance to antagonist value b.

According to an exemplary embodiment, the electronic apparatus 20 may load the pre-stored bite opening value or the occlusal distance to antagonist value without a separate input through the user interface device 23, and display the occlusal distance to antagonist value or the bite opening value corresponding to the loaded bite opening value or the occlusal distance to antagonist value.

The electronic apparatus 20 may calculate and display the occlusal state and the occlusal distance to antagonist effective for the splint manufacturing while reducing cost and time through steps S10 to S70, thereby efficiently providing the distance information necessary for the splint manufacturing to the user.

The method for processing an image according to an exemplary embodiment of the present invention may be implemented in a form of program commands that may be executed through various computer means and may be recorded in a computer-readable recording medium. In addition, an embodiment of the present disclosure may be a computer-readable recording medium on which one or more programs including commands for executing an image processing method are recorded.

The computer-readable medium may include a program command, a data file, a data structure, or the like, or a combination thereof. The program commands recorded in the computer-readable recording medium may be especially designed and configured for the present disclosure or be known to those skilled in a field of computer software. Examples of the computer-readable recording medium may include a magnetic medium such as a hard disk, a floppy disk, or a magnetic tape; an optical medium such as a compact disk read only memory (CD-ROM) or a digital versatile disk (DVD); a magneto-optical medium such as a floptical disk; and a hardware device specially configured to store and execute program commands, such as a ROM, a random access memory (RAM), a flash memory, or the like. Examples of the program commands include high-level language codes capable of being executed by a computer using an interpreter, or the like, as well as machine language codes made by a compiler. Here, the machine-readable storage medium may be provided in a form of a non-transitory storage medium.

Here, the “non-transitory storage medium” means that the storage medium is a tangible device, and does not include a signal (for example, electromagnetic waves), and the term does not distinguish between the case where data is stored semi-permanently on a storage medium and the case where data is temporarily stored thereon. For example, the “non-transitory storage medium” may include a buffer in which data is temporarily stored.

According to an embodiment, the methods according to various embodiments disclosed in the document may be included in a computer program product and provided. The computer program product may be traded as a product between a seller and a purchaser. The computer program product may be distributed in the form of a machine-readable storage medium (for example, compact disc read only memory (CD-ROM)), or may be distributed through an application store (for example, Play Store™) or may be directly distributed (for example, download or upload) between two user devices (for example, smart phones) online. In a case of the online distribution, at least some of the computer program products (for example, downloadable app) may be at least temporarily stored in a machine-readable storage medium such as a memory of a server of a manufacturer, a server of an application store, or a relay server or be temporarily created.

Although exemplary embodiments of the present disclosure have been described in detail hereinabove, the scope of the present disclosure is not limited thereto, but may include several modifications and alterations made by those skilled in the art using a basic concept of the present disclosure as defined in the claims.

1. A method for processing an image, comprising:
receiving an intraoral image including maxillary scan data and mandibular scan data;
calculating an occlusal distance to antagonist (distance to antagonist) of the intraoral image based on an occlusal state of the intraoral image; and
adjusting the occlusal distance to antagonist corresponding to the occlusal state of the intraoral image based on the calculating.

2. The method of claim 1, further comprising:
receiving the occlusal distance to antagonist for the intraoral image; and
displaying the occlusal state of the intraoral image corresponding to the received occlusal distance to antagonist.

3. The method of claim 1, further comprising:
receiving the occlusal state of the intraoral image; and
displaying the occlusal distance to antagonist for the intraoral image corresponding to the received occlusal state.

4. The method of claim 1, wherein:
the calculating includes:
calculating a first occlusal distance to antagonist corresponding to a first occlusal state of the intraoral image and a second occlusal distance to antagonist of the intraoral image corresponding to a second occlusal state different from the first occlusal state;
adjusting an occlusal state investigation range based on the first occlusal distance to antagonist and the second occlusal distance to antagonist; and
calculating an intermediate occlusal distance to antagonist for an intermediate occlusal state within the occlusal state investigation range.

5. The method of claim 4, wherein:
the calculating further includes confirming an investigated occlusal state requiring a calculation of the occlusal distance to antagonist within the occlusal state investigation range.

6. The method of claim 5, wherein:
the calculating further includes
repeating adjusting the occlusal state investigation range and calculating the intermediate occlusal distance to antagonist when there is the investigated occlusal state.

7. The method of claim 4, wherein:
the calculating further includes
extracting a tooth region in the intraoral image to extract a maxillary tooth region for the maxillary scan data and a mandibular tooth region for mandibular scan data.

8. The method of claim 7, wherein:
the calculating of the second occlusal distance to antagonist for the second occlusal state includes generating a plurality of intersections where a straight line extending in a normal direction in a plurality of meshes in the mandibular tooth region contacts the mandibular tooth region, calculating a plurality of distance values between the plurality of intersections corresponding to each of the plurality of meshes, and selecting the minimum of the plurality of distance values.

9. The method of claim 4, wherein:
the first and second occlusal states and the intermediate occlusal state are calculated in a form of bite opening.

10. The method of claim 9, wherein:
the first occlusal state corresponds to a state in which the intraoral image is in an occlusal contact relationship.

11. An electronic apparatus, comprising:
a user interface device;
a processor; and
a memory configured to store instructions executable by the processor,
wherein the processor is configured to execute the instructions to:
receive an intraoral image including maxillary scan data and mandibular scan data;
calculate an occlusal distance to antagonist (distance to antagonist) of the intraoral image based on an occlusal state of the intraoral image; and adjust the occlusal distance to antagonist corresponding to the occlusal state of the intraoral image based on the calculation operation.

12. The electronic apparatus of claim 11, wherein:
the processor is configured to receive an occlusal distance to antagonist for the intraoral image, and
display the occlusal state of the intraoral image corresponding to the received occlusal distance to antagonist.

13. The electronic apparatus of claim 11, wherein:
the calculation operation includes:
calculating a first occlusal distance to antagonist corresponding to a first occlusal state of the intraoral image and a second occlusal distance to antagonist of the intraoral image corresponding to a second occlusal state different from the first occlusal state,
adjusting an occlusal state investigation range based on the first occlusal distance to antagonist and the second occlusal distance to antagonist, and
calculating an intermediate occlusal distance to antagonist for an intermediate occlusal state within the occlusal state investigation range.

14. The electronic apparatus of claim 13, wherein:
the calculation operation further includes confirming an investigated occlusal state requiring calculation of the occlusal distance to antagonist within the occlusal state investigation range.

15. The electronic apparatus of claim 14, wherein:
the calculation operation further includes repeating adjusting the occlusal state investigation range and calculating the intermediate occlusal distance to antagonist when there is the investigated occlusal state.

16. The electronic apparatus of claim 13, wherein:
the calculation operation further includes
extracting a tooth region in the intraoral image to extract a maxillary tooth region for the maxillary scan data and a mandibular tooth region for the mandibular scan data.

17. The electronic apparatus of claim 16, wherein:
the calculating of the second occlusal distance to antagonist for the second occlusal state includes generating a plurality of intersections where a straight line extending in a normal direction in a plurality of meshes in the mandibular tooth region contacts the mandibular tooth region, calculating a plurality of distance values between the plurality of intersections corresponding to each of the plurality of meshes, and selecting the minimum of the plurality of distance values.

18. The electronic apparatus of claim 13, wherein:
the first and second occlusal states and the intermediate occlusal state are calculated in a form of bite opening.

19. The electronic apparatus of claim 18, wherein:
the first occlusal state corresponds to a state in which the intraoral image is in an occlusal contact relationship.

20. A computer readable storage medium including computer readable instructions,
wherein the instructions cause the computer to:
receive an intraoral image including maxillary scan data and mandibular scan data;
calculate an occlusal distance to antagonist of the intraoral image based on an occlusal state of the intraoral image; and
adjust the occlusal distance to antagonist corresponding to the occlusal state of the intraoral image based on the calculating.
